(19) United States
(12) Patent Application Publication
(10) Pub. No.: US 2023/0110647 A1
(43) Pub. Date: Apr. 13, 2023

(54) MULTI-MODULUS PROBE DESIGN AND ASSEMBLY

(71) Applicant: Northgate Technologies Inc., Elgin, IL (US)

(72) Inventors: Alexander Carlo Buscaglia, Franklin, IN (US); Christian Lupercio, Carpentersville, IL (US); Alexander Travis Brown, Denver, CO (US)

(73) Assignee: Northgate Technologies Inc., Elgin, IL (US)

(21) Appl. No.: 17/888,667

(22) Filed: Aug. 16, 2022

Related U.S. Application Data
(60) Provisional application No. 63/233,633, filed on Aug. 16, 2021.

(51) Int. Cl. A61B 17/22

(52) U.S. Cl. CPC A61B 17/22022; A61B 2017/00862

(57) ABSTRACT

A multi-modulus probe design and assembly are disclosed. In one implementation, the system may include a stiffening element extending along at least a portion of a length of a lumen containing electrical wires for electrodes used in an Electrohydraulic Lithotripsy (EHL) probe. The stiffening element may be contained within the lumen of the probe or may extend along an outside of the probe. The stiffening element may be an assembly of wires of different modulus of elasticity that are joined together with a non-conductive sheath such that a greater stiffness is provided at a proximal end, and a lesser stiffness is provided at a distal end near a tip of the probe.

### CROSS-REFERENCE TO RELATED APPLICATIONS

This application claims the benefit of U.S. Application Serial No. 63/233,633, filed Aug. 16, 2021, pending, the entirety of which is hereby incorporated herein by reference.

### FIELD OF THE DISCLOSURE

The present disclosure relates to electrical probe assemblies such as those used in electrohydraulic lithotripsy (EHL) systems for biliary or urological stone management.

### BACKGROUND

Stone disease is a painful and common condition that affects numerous adults in the US. A large number of doctor or emergency room visits happen every year because of kidney stones. Larger stones may cause obstruction in the gallbladder, bile duct, kidney, ureter and bladder. If needed, lithotripsy or surgical techniques may be used for stones which do not pass through the bile duct, ureter or bladder on their own. Electrohydraulic Lithotripsy (EHL) is primarily used in endoscopic procedures to fragment large and difficult biliary (bile duct or gallbladder) and renal calculi (kidney, urinary, bladder) stones.

Electrical probes used in EHL systems are powered with a generator that supplies power to a tip of the probe. The tip of the probe will receive a charge that results in a spark that generates a shock wave to mechanically break up stones in a body. These probes are often inserted into an applicable visualization scope and passed through the working channel to reach the problem stone. One common component in the assembly of the probes used with EHL is a polyimide tube that encases the long central wires that carry the electrical charge of the probe during use. The tube is very thin and flexible, however its integrity during passage through the scope, as well as along tight curves, can be compromised.

To gain access to the common bile duct or an upper pole of a kidney, an endoscope is typically used for visualization. The endoscope has a proximal end that the surgeon holds and controls the distal end with, and a distal end that can be controlled for orientation and positioning. This distal end can be curved to allow for optimal clinical access to surgical site. The endoscope, which has digital visualization, is typically placed in the correct orientation before a probe is passed through its working channel. The curvatures of a working channel can be difficult for a probe to pass through due to the differing needs to properly pass the device. The probe must be rigid enough to allow for pushing force on the proximal end but also flexible enough to bend along the tight curves of the distal end of the scope. If forced through, the outer polyimide tubing can kink, either at the scope working channel entry point where the user is feeding it or at the distal end where the probe is stuck and attempted to be forced along the curved path, causing issues with continued functionality.

### SUMMARY

In order to improve the ability of a probe to maintain flexibility for insertion and integrity for functionality, a multi-modulus probe design is disclosed herein.

According to one aspect, a multi-modulus wire assembly for use in a lithotripsy probe is described. The multi-modulus wire assembly may include a first wire having a first outer diameter and a second wire having a second outer diameter, where the second outer diameter is less than the first outer diameter. The wire assembly may also include a non-conductive connecting sheath positioned on, and connecting, a first end of the first wire and a first end of the second wire, where an inner diameter of the non-conductive connecting sheath is substantially equal to the first outer diameter. An adhesive may be positioned in the non-conductive connecting sheath connecting the first and second wires together with the non-conductive connecting sheath. The modulus of elasticity of the multi-modulus wire assembly is greater at a first location along the first wire than at a second location along the second wire.

According to another aspect, a lithotripsy probe, the lithotripsy probe may include an elongate body having a proximal end and a distal end, wherein the elongate body defines a lumen extending from the proximal end to the distal end. The lithotripsy probe may further include a stiffening assembly positioned within the lumen of the elongate body, the stiffening assembly extending from the proximal end to the distal end of the elongate body; and. Additionally, the stiffening assembly comprises a variable stiffness having a greater stiffness at the proximal end than the distal end of the elongate body. In different embodiments, the stiffening assembly may include first and second wires of different outer diameters and different modulus of elasticity attached via a non-conductive sheath.

In yet another aspect of a lithotripsy probe, a lithotripsy probe may include an elongate body having a proximal end and a distal end, wherein the elongate body defines a lumen extending from the proximal end to the distal end. In addition, the probe may include first and second conductive wires positioned inside, and extending along, the lumen of the elongate body, wherein the first and second conductive wires are configured to receive an electric charge from a source positioned at the proximal end of the elongate body and generate a shockwave at a tip positioned at the distal end of the elongate body. Further, a stiffening assembly may be positioned along the lumen of the elongate body, the stiffening assembly comprising an outer sheath of the lumen, the outer sheath comprising a multi-layer material extending from the proximal end to the distal end of the elongate body, where the stiffening assembly has a variable stiffness that is greater at the proximal end than the distal end of the elongate body.

Other embodiments are contemplated and described herein.

### BRIEF DESCRIPTION OF THE DRAWINGS

For the purpose of facilitating an understanding of the subject matter sought to be protected, there is illustrated in the accompanying drawings an embodiment thereof, from an inspection of which, when considered in connection with the following description, the subject matter sought to be protected, its construction and operation, and many of its advantages should be readily understood and appreciated.

FIG. 1 illustrates a multi-modulus wire for use in an EHL probe according to an embodiment of the present invention.

FIG. 2 is a cross-sectional view of the multi-modulus wire of FIG. 1.

FIG. 3 is a simplified sectional view of an example EHL probe, without the functional electrode wires illustrating an internal multi-modulus wire embodiment.

FIG. 4 shows the EHL probe of FIG. 3 with additional internal wires and structures in addition to the internal multi-modulus wire.

FIG. 5 is a sectional view of the proximal end an EHL probe having an internal stiffening element of a multi-modulus wire.

FIG. 6 shows a cross-sectional view of the EHL probe of FIG. 5 taken along line N-N.

FIG. 7 shows a cross-sectional view of the EHL probe of FIG. 5 taken along line P-P.

FIG. 8 shows a cross-sectional view of the EHL probe of FIG. 5 taken along line R-R.

### DETAILED DESCRIPTION

The present disclosure is directed to a probe for use in electrohydraulic lithotripsy (EHL) applications that has multi-modulus features to allow variable stiffness and maneuverability of the probe along its length. In one embodiment, a multi-modulus stiffener may be applied to a part of the probe to allow for varying levels of stiffness along the length of the probe while consistently applying additional reinforcement to the fully-assembled product. Thus, one version of a multi-modulus probe disclosed herein allows for a substantially reinforced section, while another section may be only slightly reinforced to allow custom flexibility to the extent and at the locations needed for optimal use for a particular application.

In a standard EHL probe, a first electrode and a second electrode are positioned within, and extend longitudinally and electrically isolated from one another, along the interior region of the EHL probe. The majority of the length of the probe is typically covered by an insulating sheath, such as polyimide, to electrically isolate the electrodes from the exterior of the probe. As known in the art, at a proximal end of the EHL probe the first and second electrodes are coupled with an electrical source, such as an electrohydraulic generator (for example the AUTOLITH® generators, supplied by Northgate Technologies Inc. of Elgin, Illinois) is used to charge the first electrode to one polarity and the second electrode to an opposite polarity. When the first electrode is charged to a first polarity and the second electrode is charged to a second, opposite polarity, a discharge of electricity occurs at or adjacent the tip located at the distal end of the EHL probe between the first and second electrodes (an electrical arc) when the potential between the first and second electrodes reaches the dielectric breakdown voltage for the media separating the electrodes.

Embodiments disclosed herein include a stiffener for an EHL probe for ease and effectiveness of use. The stiffened EHL probe may be fabricated by adding a multi-modulus wire assembly, also referred to herein as a stiffening element or stiffening assembly, that is added to fit within the existing design diameter of an EHL probe in order to provide varying moduli of elasticity along the length of the probe. This varying elasticity may avoid weakness in the probe that can lead to kinking or other problems in standard probes. In one implementation, the multi-modulus wire assembly may be designed to fit within the existing design diameter of an EHL probe such that the outer sheath of the EHL probe contains all of the electrodes and the multi-modulus wire assembly. In this modified EHL probe, the multi-modulus wire is constructed and then inserted into the outer polyimide sheath during assembly, similar to the application of the center and ground wires.

The internalization of the multi-modulus wire assembly into an existing EHL probe sheath can allow for a seamless exterior profile of the probe and avoid introducing any other modes of failure when assessing usability with clinical devices (such as endoscopes and so on). In this embodiment, the multi-modulus wire assembly may be separate from the electrodes, however other embodiments are contemplated where the multi-modulus wire assembly may also serve as part of an electrode. In yet other implementations, it is contemplated that the multi-modulus wire assembly may be applied externally to the sheath of the EHL probe. It is expected that applying a multi-modulus assembly of materials to an existing EHL probe may reinforce the construction and aid in the prevention of kinks/breaks of the shaft during clinical use regardless of whether the materials are fabricated with the rest of the EHL probe as an internal component or an external component.

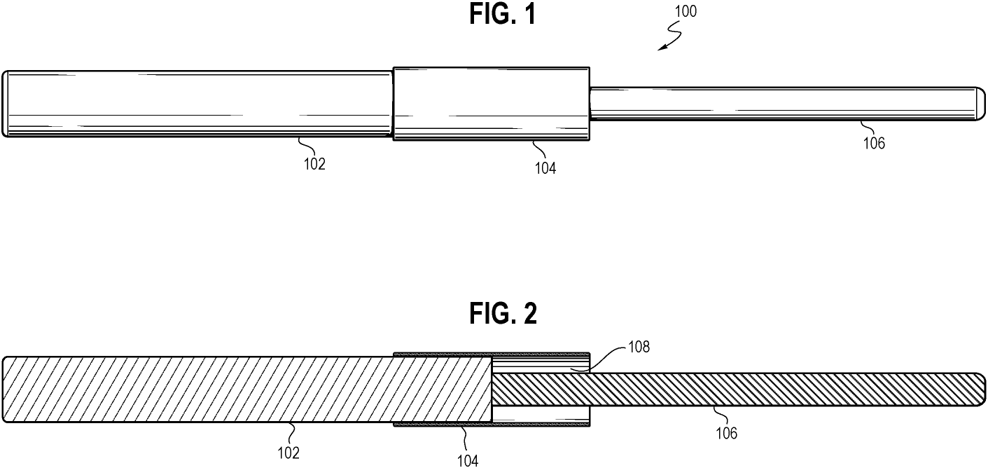

As shown in FIGS. 1-2, one example of a multi-modulus wire assembly 100 that may be integrated into existing EHL probes is shown. The multi-modulus wire assembly 100 may be made up of an assembly of materials with varying moduli of elasticity. The multi-modulus wire assembly 100 may include a first wire 102 having a first outer diameter (OD) that is connected with a second wire 106 having an OD less than the first OD. The first and second wires 102, 106 may be connected via a sheath 104 that has an OD slightly greater than the first OD. The sheath 104 may be made from a polyamide material, or other non-conductive material, and extends over the ends of both the first and second wires 102, 106. As seen in FIG. 2, which is a cross-section of the multi-modulus wire assembly 100 of FIG. 1, the sheath may be filled with an adhesive compound 108 to hold the wires 102, 106 and sheath 106 together.

The first and second wires 102, 106 may be any of a number of different flexible metals or non-metals, for example Nickel-Titanium (NiTi) alloy metal wires. Any number of differing moduli of elasticity may be achieved by connecting two or more long (for example, 50 cm-450 cm), thin (for example, 0.025 cm OD - 0.3 cm OD) materials, of varying levels of Youngs modulus (20-100 GPa) and assembled to fit within a standard EHL probe diameter range, such as 1-9 Fr (0.033 cm - 0.3 cm). For example, rather than a single stage transition as shown in FIGS. 1-2, the multi-modulus wire assembly may have multiple stages, where each stage has a larger OD wire connected to a smaller OD wire via a joint (e.g. first wire OD > second wire OD > third wire OD, and so on). Each wire may have a constant modulus of elasticity, that is different from each other wire in one implementation.

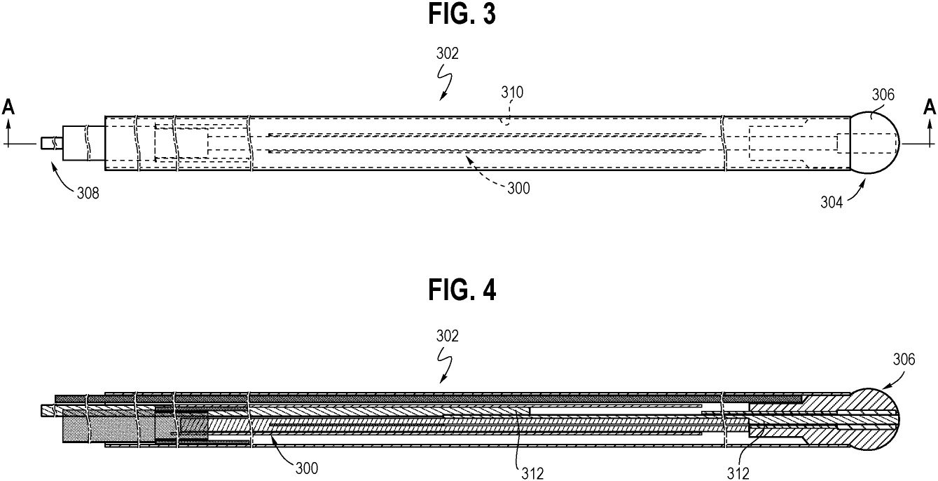

Referring now to FIGS. 3-4, an example multi-modulus wire assembly 300 similar to that of FIGS. 1-2 is shown as part of a larger EHL probe package 302 having a proximal end 308 where conductors for the electrodes may be connected to a generator, and a distal end 304 where a tip 306 designed to direct the shock wave from a generated spark is located. In FIG. 3, other internal components (e.g. the additional electrode wires/structures) are not shown for ease of view of the internal stiffener (multi-modulus wire assembly 300). In FIG. 4, the EHL probe of FIG. 3 is shown in cross-section, but with an example set of internal components adjacent the multi-modulus wire assembly 300 inside the outer sheath 310 of the EHL probe 302 for context. Example conductive wires 312 of the electrodes are shown that, as described above, carry differing charges supplied by a generator to produce a spark and associated shock wave at the tip 306.

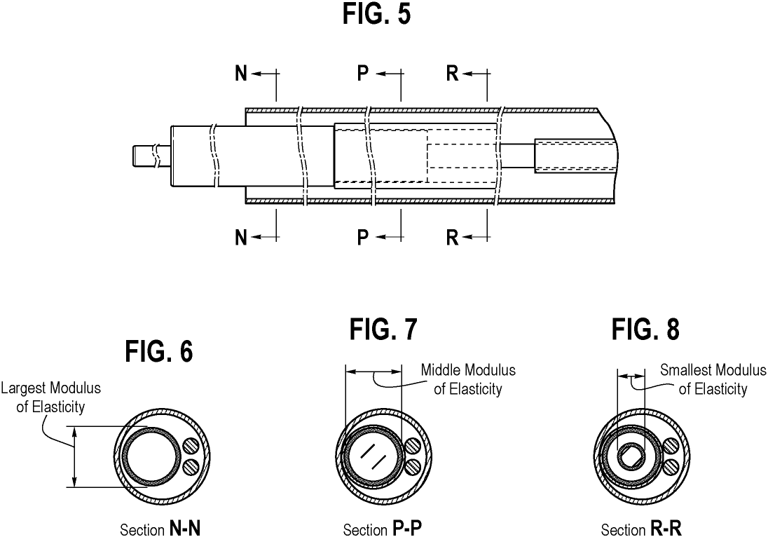

Referring now to FIGS. 5-8, FIG. 5 shows a sectional view of the proximal end of an EHL probe, such as shown in FIG. 3, and FIGS. 6-8 illustrate cross sections of FIG. 5 taken at lines N-N, P-P and R-R of FIG. 5, respectively. In the cross-section shown in FIG. 6, two electrodes are shown adjacent the multi-modulus wire. The multi-modulus wire material (which may be a NiTi wire, sometimes referred to as Nitinol, or other wire material) is the larger OD wire is the only internal stiffener for the EHL probe and provides the largest modulus of elasticity (i.e. is at its stiffest).

The second cross-section shown in FIG. 7, spaced closer toward the distal end of the EHL probe, illustrates a point along the multi-modulus wire assembly where the joining zone of the multi-modulus wire system is located. This location is thus where the larger OD NiTi wire is ground down to allow for easier assembly with the smaller OD NiTi wire and the bonding sheath such that it provides a middle modulus of elasticity that is less than that of the portion of the multi-modulus wire assembly shown in FIG. 6. Finally, a smallest modulus of elasticity (i.e., least stiff) for the three illustrated EHL probe cross-sections the EHL probe is provided at the cross-section shown in FIG. 8, where a smaller OD diameter wire, smaller than the larger od wire than that of FIG. 6 is the only internal stiffener.

In one embodiment, in order to assemble the different OD wires and create a multi-modulus wire assembly such as described above, each of the different OD wires may be coated in a non-conductive coating and the ends of the different OD wires to be connected may first be stripped of the coating. The wire coating of each original NiTi, or other type of, wire may be stripped for a predetermined distance, for example 0.25 to 1.0 inches, from the end of each wire. The original coating to be stripped may be a polyimide or other standard coating. Alcohol may then be applied to the bare metal of the wire and then dried off. The bare ends of the stripped wires of differing OD that are to be joined may each then slipped into opposite ends of a new sheath, for example of polyimide. The new sheath may be of a diameter that snugly fits over the bare wire of the larger OD piece of wire and an adhesive may be added as the pieces are slid together. The sheath may be slid over all of the stripped wire up to the end of the remaining original coating of that wire so that no bare wire of either the larger OD or smaller OD wires is exposed. On the opposite end of the sheath, the stripped end of the smaller OD wire and a larger amount of the same adhesive may be positioned in the sheath. The length of the sheath covering and joining the ends of the different OD wires may be sufficient to completely cover all bare metal of the stripped ends of the wires. Any of a number of adhesives may be used between the sheath and the wires. One suitable adhesive may be a LOCTITE brand epoxy (e.g. cyanoacrylate glue) available from Henkel Corp. of Westlake, Ohio.

In another embodiment, in addition to, or separate from, the embodiment above of different OD wires and connecting sheaths, other variable stiffness EHL probes are contemplated. For example, application of a multi-layer material to an EHL probe may be used to reinforce the probe along its length to various levels, with the option of varying the layer’s density. In one implementation, utilization of a polyimide tube as the outer sheath of the EHL probe (OD from 0.033 cm-0.3 cm) comprised of at least three layers of material that has a braided matrix material (0.5 x 3 mil stainless steel, or another material of like properties and size) as one of the layers. This braided material (ranging from 30- 120 PPI, where PPI is used herein to refer to pics per inch and “pic” is per inch crosses or the density of the braid) could be consistent all the way through the polyimide or it could vary in the density of the braid along its length. This material may allow for an increase in the longitudinal rigidity in comparison to a standard polyimide tube anywhere from 5-200%. For example, current standard polyimide with an outer diameter of 0.0316 inches has a longitudinal rigidity of approximately 130 lbf. Addition of the braided material may increase longitudinal rigidity to approximately 135 lbf - 300 lbf. The addition of the braided material to an EHL probe that has polyimide as its outermost layer may reduce the prevalence of kinking or malfunction of the probe during insertion or clinical use. This embodiment of adding a braded material to the outer sheath of the EHL probe may allow the probe’s outer sheath to maintain its dielectric strength (a requirement in its current form) as well as its dimensional constraints. This also may allow the probe to be reinforced and strengthened along its length without the need for a full redesign of the internal components, nor a reassessment of the scopes with which the EHL probe is expected to function with.

In addition to a uniform addition of braided material to the outer sheath of the EHL probe that has polyimide as its outermost layer, the addition of the braiding material referenced above in a varied manner in terms of braiding density to an EHL probe is contemplated. The variation in braiding may allow for custom strengthening, increase in rigidity and/or increase in flexibility, to certain sections of the EHL probe which would allow for the passage of the probe through tight anatomies to be performed without damage to the probe. As an example, the braiding could be less dense (stiffer) on the proximal end of the probe and more dense (more flexible) on the distal end of the probe leading to easier pushability of the probe due to the stronger proximal end, as well as easier passability due to the leading edge of the probe nearer the tip being more flexible.

In yet another alternative embodiment, rather than including a multi-modulus wire assembly inside the outer sheath of the EHL probe, it is contemplated that the EHL probe may instead be fabricated with a single wire or a multi-modulus wire in positioned along the polyimide outer sheath. In one embodiment the single or multi-modulus wire would maybe located between the layers of the multi-layer polyimide described above and may add even more rigidity (based on material type and size) to the reinforced braiding embodiment. In another embodiment, the multi-modulus wire assembly may be attached to the exterior of the outer polyimide sheath of the EHL probe.

Although the present invention has been described with reference to several exemplary embodiments, those skilled in the art will recognize that changes may be made in form and detail without departing from the spirit and scope of the invention. As such, it is intended that the foregoing detailed description be regarded as illustrative rather than limiting and that it is the appended claims, including all equivalents thereof, which are intended to define the scope of the invention.

We claim:

1. A multi-modulus wire assembly for use in a lithotripsy probe, the multi-modulus wire assembly comprising:
a first wire having a first outer diameter;
a second wire having a second outer diameter, wherein the second outer diameter is less than the first outer diameter;
a non-conductive connecting sheath positioned on, and connecting, a first end of the first wire and a first end of the second wire, wherein an inner diameter of the non-conductive connecting sheath is substantially equal to the first outer diameter;
an adhesive positioned in the non-conductive connecting sheath connecting the first and second wires together with the non-conductive connecting sheath; and
wherein a modulus of elasticity of the multi-modulus wire assembly is greater at a first location along the first wire than at a second location along the second wire.

2. The multi-modulus wire assembly of claim 1, further comprising:
a third wire having a third outer diameter, wherein the third outer diameter is less than the second outer diameter;
a second non-conductive connecting sheath positioned on, and connecting, a second end of the second wire and a first end of the third wire, wherein an inner diameter of the second non-conductive connecting sheath is substantially equal to the second outer diameter; and
the adhesive positioned in the second non-conductive connecting sheath connecting the second and third wires together with the second non-conductive connecting sheath.

3. A lithotripsy probe, the lithotripsy probe comprising:
an elongate body having a proximal end and a distal end, wherein the elongate body defines a lumen extending from the proximal end to the distal end;
a stiffening assembly positioned within the lumen of the elongate body, the stiffening assembly extending from the proximal end to the distal end of the elongate body; and
wherein the stiffening assembly comprises a variable stiffness having a greater stiffness at the proximal end than the distal end of the elongate body.

4. The lithotripsy probe of claim 3, wherein the lithotripsy probe is an electrohydraulic lithotripsy probe and further comprises:
first and second conductive wires extending through the lumen of the elongate body, wherein the first and second conductive wires are configured to receive an electric charge from a source positioned at the proximal end of the elongate body and generate a shockwave at a tip positioned at the distal end of the elongate body.

5. The lithotripsy probe of claim 3, wherein:
the stiffening assembly further comprises:
a first section having a first outer diameter;
a second section having a second outer diameter, wherein the second outer diameter is less than the first outer diameter;
a connecting sheath positioned on, and connecting, a first end of the first section and a first end of the second section, wherein an inner diameter of the connecting sheath is substantially equal to the first outer diameter; and
wherein a modulus of elasticity of the stiffening assembly is greater at a first location along the first section than at a second location along the second section.

6. The lithotripsy probe of claim 5, further comprising an adhesive positioned in the connecting sheath connecting the first and second sections together with the connecting sheath.

7. The lithotripsy probe of claim 5, wherein the first section and the second section are formed of respective wires.

8. The lithotripsy probe of claim 7, wherein the respective wires are NiTi wires.

9. The lithotripsy probe of claim 5, wherein the connecting sheath comprises a non-conductive material.

10. A lithotripsy probe, the lithotripsy probe comprising:
an elongate body having a proximal end and a distal end, wherein the elongate body comprises an outer sheath of non-conductive material that defines a lumen extending from the proximal end to the distal end;
a stiffening assembly positioned on the outer sheath of the elongate body, the stiffening assembly extending from the proximal end to the distal end of the elongate body; and
wherein the stiffening assembly comprises a variable stiffness having a greater stiffness at the proximal end than the distal end of the elongate body.

11. The lithotripsy probe of claim 10, wherein the lithotripsy probe is an electrohydraulic lithotripsy probe and further comprises:
first and second conductive wires extending through the lumen of the elongate body, wherein the first and second conductive wires are configured to receive an electric charge from a source positioned at the proximal end of the elongate body and generate a shockwave at a tip positioned at the distal end of the elongate body.

12. The lithotripsy probe of claim 11, wherein:
the stiffening assembly further comprises:
a first section having a first outer diameter;
a second section having a second outer diameter, wherein the second outer diameter is less than the first outer diameter;
a connecting sheath positioned on, and connecting, a first end of the first section and a first end of the second section, wherein an inner diameter of the connecting sheath is substantially equal to the first outer diameter; and
wherein a modulus of elasticity of the stiffening assembly is greater at a first location along the first section than at a second location along the second section.

13. The lithotripsy probe of claim 12, further comprising an adhesive positioned in the connecting sheath connecting the first and second sections together with the connecting sheath.

14. The lithotripsy probe of claim 12, wherein the first section and the second section are formed of respective wires.

15. The lithotripsy probe of claim 14, wherein the respective wires are NiTi wires.

16. A lithotripsy probe, the lithotripsy probe comprising:
an elongate body having a proximal end and a distal end, wherein the elongate body defines a lumen extending from the proximal end to the distal end;
first and second conductive wires positioned inside, and extending along, the lumen of the elongate body, wherein the first and second conductive wires are configured to receive an electric charge from a source positioned at the proximal end of the elongate body and generate a shockwave at a tip positioned at the distal end of the elongate body; and
a stiffening assembly positioned along the lumen of the elongate body, the stiffening assembly comprising an outer sheath of the lumen, the outer sheath comprising a multi-layer material extending from the proximal end to the distal end of the elongate body, wherein the stiffening assembly comprises a variable stiffness having a greater stiffness at the proximal end than the distal end of the elongate body.

17. The lithotripsy probe of claim 16, wherein the multi-layer material comprises at least three layers of material and wherein one of the at least three layers comprises a braided matrix material.

18. The lithotripsy probe of claim 16, further comprising a second stiffening assembly, the second stiffening assembly positioned within the lumen of the elongate body, the stiffening assembly extending from the proximal end to the distal end of the elongate body; and
wherein the second stiffening assembly comprises a second variable stiffness having a greater stiffness at the proximal end than the distal end of the elongate body.

19. The lithotripsy probe of claim 18, wherein the second stiffening assembly further comprises:
a first section having a first outer diameter;
a second section having a second outer diameter, wherein the second outer diameter is less than the first outer diameter;
a connecting sheath positioned on, and connecting, a first end of the first section and a first end of the second section, wherein an inner diameter of the connecting sheath is substantially equal to the first outer diameter; and
wherein a modulus of elasticity of the stiffening assembly is greater at a first location along the first section than at a second location along the second section.

20. The lithotripsy probe of claim 19, wherein the first section and the second section are formed of respective wires, and further comprising an adhesive positioned in the connecting sheath connecting the respective wires together with the connecting sheath.
